(19) United States
(12) Patent Application Publication
(10) Pub. No.: US 2007/0213692 A1
(43) Pub. Date: Sep. 13, 2007

(54) FORCE ACTION FEEDBACK IN SURGICAL INSTRUMENTS

(76) Inventors: Timo Neubauer, Poing/Angelbrechting (DE); Anusch Saffari, Waakirchen (DE); Manuel Millahn, Munich (DE)

Correspondence Address: DON W. BULSON (BrainLAB), RENNER, OTTO, BOISSELLE & SKLAR, LLP, 1621 EUCLID AVENUE - 19TH FLOOR, CLEVELAND, OH, 44115, US

(21) Appl. No.: 11/684,015

(22) Filed: Mar. 9, 2007

Related U.S. Application Data
(60) Provisional application No. 60/744,221, filed on Apr. 4, 2006.

(30) Foreign Application Priority Data
Mar. 9, 2006 (EP) 06004822

(51) Int. Cl. A61B 17/00

(52) U.S. Cl. 606/1

(57) ABSTRACT

A surgical instrument includes a holding section, a tool removably attachable to the holding section, and at least one load sensor. The at least one sensor is operative to measure a mechanical load exerted by the tool on the instrument.

### RELATED APPLICATION DATA

This application claims priority of U.S. Provisional Application No. 60/744,221 filed on Apr. 4, 2006, which is incorporated herein by reference in its entirety.

### FIELD OF THE INVENTION

The invention relates to surgical instruments and, more particularly, to a device, system and method using force action feedback in surgical instruments.

### BACKGROUND OF THE INVENTION

An example of a surgical instrument is a bone saw. During use of a bone saw, the saw blade can become deformed due too significant loads placed on the saw blade, which may occur while sawing a highly resistant material. This deformation can impair the incision process itself, but also can lead to irregular incision planes. When the instrument is monitored within the framework of surgical navigation, deformations in the tool may lead to navigation errors.

DE 100 24 221 D1 proposes a solution to the problems mentioned above for a surgical saw. In accordance with this proposal, a bending or positional sensor is arranged on or in the saw blade. The sensor generates signals corresponding to its spatial configuration, and the signals are provided to a signal processing device.

One problem with this approach is that every tool, e.g., every saw blade, has to be provided with such a sensor device. This makes the tool, which may be subject to wear, expensive to purchase and manufacture. Further, tools provided with such sensors can be complicated to sterilize.

### SUMMARY OF THE INVENTION

An instrument includes at least one load sensor that measures a mechanical load exerted by a tool at the instrument. In other words, how the tool itself deforms is not directly measured. Instead, a load measurement can be made at a point where the load is transferred by the tool onto the instrument. The load exerted on the tool by an operator of the surgical instrument continues from the tool itself through the instrument. This can be used to place the measuring means (e.g., the load sensor) away from the tool itself and into any other part of the surgical instrument. It is then possible to use conventional tools such as are commercially available and which can be conventionally and easily sterilized. Further, permissible loads for these tools can be ascertained.

The load sensor can include a force or torque sensor, and such sensors can be adapted for use with a tool.

There exists the possibility of coupling the load sensor to a load indicator on the instrument, in particular on a holding section of the instrument, in order to indicate the actual load. Such a load indicator can comprise one or more load state indicators that indicate the load state, such as a color code, for example.

The instrument can comprise a tool adaptor, in particular a universal adaptor for a number of matching tools, which may be embodied as a plug or latch connection. The load sensor then can measure a mechanical load for different tools, such that the load state can always be indicated.

There exists the possibility of providing the tool with an identification means which mechanically or wirelessly transmits information about the nature of the tool to a receiver on the instrument (e.g., on a holding section of the instrument). In such an embodiment, the identification means can be an RFID (radio-frequency identification) transponder, wherein the receiver is an RFID receiver or an RFID transmitter/receiver unit.

Using the identification technology, tool-specific load states then can be measured and indicated. The load which may be allowed for a particular tool without tool deformation or otherwise functionally impairing the tool can be ascertained beforehand. If the load indicator is then suitably adapted for specific tools, it is possible to indicate for each tool whether it is being used within a permissible load range or whether it is being overloaded.

The surgical instrument can include a tool drive, e.g., a so-called “power tool”. In such an instrument, the load sensor can be arranged at a point in the drive chain of the tool. In very general terms, but also for power tools, it is possible to arrange the load sensor on the tool adaptor or on a drive member including the tool adaptor. The load sensor also can be situated on a drive-transmitting coupling assigned to the holding section of the instrument.

The surgical tool may be a surgical saw that is electrically or pneumatically operated, such as a bone saw, for example, that includes an oscillating saw blade. It should be noted that in principle, the invention can be used with any surgical instruments in which loads are exerted via a tool and in which it is advantageous to monitor such loads.

In accordance with another aspect of the invention, a medical instrument monitoring system includes a surgical instrument such as has been described herein, wherein the instrument includes a transmission unit that transmits a load state ascertained by a load sensor to a receiver assigned to a data processing unit. In other words, the load state is relayed and processed in accordance with the instrument monitoring system. This can be advantageous, for example, wherein a data processing unit includes an image output on which the load state can be provided. It is often advantageous if the load state is not output on the instrument itself, but rather on a separate image output that can provide a larger display with better resolution (e.g., on a monitoring or navigation screen in an operating theater). Additionally, acoustic signals also can be output that audibly indicate an overload state.

The data processing unit can be assigned to a medical tracking and navigation system that determines and tracks the position of the instrument. In this case, the tracking and navigation system can include an evaluation unit that correlates the load state and the movement of the instrument, and ascertains data about necessary or possible adaptations of the movement or operation. This data then can be provided to the image output.

The method features cited here and/or the implementation, as a method, of the features by which the device has been described naturally form part of the overall disclosure of the invention. The device, system and method described herein improves and simplifies surgical procedures performed using load-sensitive tools (e.g., sawing bones in total knee replacements). When the indicator and the identification features are utilized, the system can determine (e.g., due to the information that it receives from the transponder, such as material data and geometry data) the maximum force that is allowed without deformation of the tool. The information from the sensor can be combined with the information from the navigation system so as to provide the user with a precise indication of the depth of the incision by the tool, and this can also be used to adapt the speed of the tool to the density of the tissue currently being treated.

Using the device, system and method described herein, the surgeon can receive intra-operative information about forces acting on the tool. This feedback gives the surgeon the option of detecting and reducing high pressure on the instrument and, thus, reducing the risk of tool deformation. It is then possible to more precisely prepare a bone, such that implants (e.g., knee replacement implants) exhibit improved fit on the patient. If a transponder system (RFID) is used, the software can automatically ascertain tool-specific information (such as geometric data). This reduces the risk of calibration errors and makes the use of navigated instruments easier. A navigated surgical instrument provides precise data about the depth of incision, and the data also can be used to adjust the drive (speed) of the tool to the density of the tissue currently being treated or about to be treated.

### BRIEF DESCRIPTION OF THE DRAWINGS

The forgoing and other features of the invention are hereinafter discussed with reference to the drawings.

FIG. 1 is a side view of an exemplary surgical instrument embodied as a saw in accordance with the invention.

FIG. 2 is a detail view of the surgical instrument of FIG. 1 with a load sensor array in accordance with the invention.

FIG. 3 is a section view of a portion of an exemplary saw attachment coupling in accordance with the invention.

FIG. 4 is a detail section view of an exemplary drill attachment coupling with axial force measured in accordance with the invention.

FIG. 5 is a detail view of an exemplary saw attachment coupling with force measured in the clamp in accordance with the invention.

### DETAILED DESCRIPTION

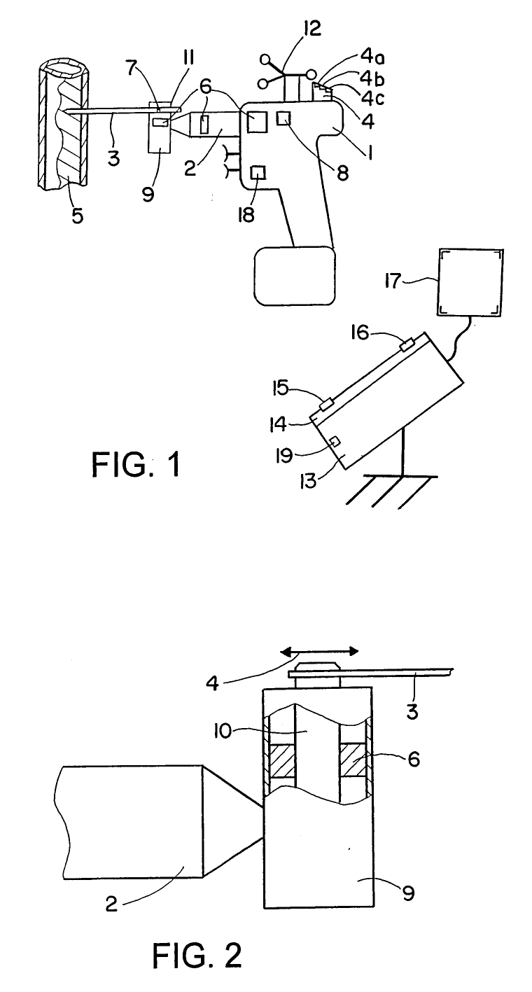

With reference to FIG. 1, a holding section or handle 1 of a surgical saw is shown, wherein the saw includes a rotational coupling 2. Attached to a front or end portion of the coupling 2 is a tool holder 9. The tool holder 9 can oscillate rotationally and can include a tool adaptor 11, wherein a saw blade 3 may be inserted into the tool adapter 11. The saw blade 3 can include an RFID transponder 7 that transmits information about the tool (e.g., information about the saw blade 3) to an RFID transmitter/receiver 8. The information can identify the tool 3 with respect to its physical features, such as, for example, a length and/or position of the functional section (e.g., a part of the saw blade which actually cuts). The transponder 7, however, also can transmit data regarding other features of the saw blade 3. This other information can include, for example, a permissible load that can occur during the sawing process without deformation of the saw blade 3.

The actual applied load, e.g., the load that the saw blade 3 exerts on the tool holder 9, is ascertained by a load sensor 6 (e.g., a force or torque sensor) attached to the tool holder 9 and/or to the coupling 2 (both positions are shown in FIG. 1). This determined load then can be transmitted (by cable or wirelessly) with the aid of a data transmitter (not shown) to a data processing unit (e.g., in the holding section 1), which can correlate the actual load state with permissible load states, and a signal then can be output that indicates the load state to the user.

One way of outputting a signal is to use a load state indicator 4, which in the exemplary device of FIG. 1 includes three LEDs 4a, 4b and 4c. Various possibilities then present themselves. For example, an overload can be indicated by all three LEDs being energized. Alternatively, the LEDs can be color-coded, for example, green, yellow and red for a normal, increased and overload state. The user then can adapt the force he exerts when sawing a bone 5 having a harder outer layer and a softer core, depending on the load state indicated.

FIG. 2 shows another exemplary tool wherein the saw blade 3 is placed on and secured to the end of an oscillating transmission shaft 10. The load sensor 6 can be situated on the tool holder 9, which is placed on the coupling 2 at a slightly higher point, and engages with shaft 10. The load sensor 6 can absorb and relay larger or smaller forces or torques, depending on where it is arranged. In the exemplary tool of FIG. 2, the sensor 6 can measure the inclination of the oscillating shaft 10, indicated by the arrow 4. In principle, however, the load sensor 6 could also be provided in the rotational coupling 2 or in components connected to the rotational coupling 2 and/or the load sensor 6, depending on how directly the load is measured.

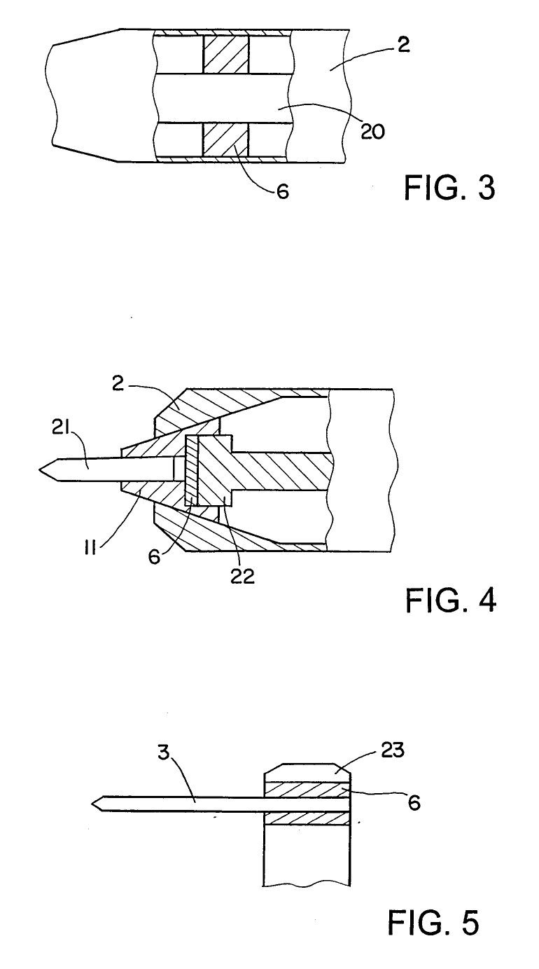

FIGS. 3, 4 and 5 show other possible arrangements for the load sensor 6. FIG. 3 shows a detail of an attachment coupling 2 for a power tool that can be used with the saw tool. In this embodiment, the sensor 6 can be arranged on a drive shaft 20 (e.g., around the shaft 20 in the casing of the coupling 2) and can measure a rotation of the shaft 20 and the attendant stresses. FIG. 4 shows a detail of a drill attachment for a power tool. The tool holder 11 can be mounted in the coupling 2 and can hold a drill bit 21, for example. The sensor 6 can be mounted between a transmission bolt and the tool holder 11, and can measure an axial force exerted on the clamping jaws of the tool holder 11. Lastly, FIG. 5 shows another method of measuring a force, wherein a sensor 6 is accommodated in a saw blade holder 23. The saw blade 3 can be directly held by the sensor 6 such that it can measure the force that occurs at the jaws holding or clamping saw blade 3. Thus, the sensor 6 itself serves as a clamp for a tool.

Returning now to FIG. 1, it may be seen that the bone saw shown in FIG. 1 is used in a surgical navigation environment. To this end, it can include an optically detectable reference star 12, the position of which can be ascertained by a navigation/tracking system 13. The navigation/tracking system 13 can include a tracking/camera unit 14 comprising cameras 15, 16, which can ascertain a position of the surgical instrument via the reference star 12 and, therefore, also a position of the saw blade 3 and/or its tip. In particular, this is possible when the surgical instrument, together with its data and the information about the physical features of the saw 3, are stored in the memory of the navigation system 13 (e.g., a pre-calibration or pre-calibrated instrument).

One task of the navigation system 13 may be to track a position of the surgical instrument, e.g., the surgical saw, and to correlate these positional data with body structure data (CT, MR) ascertained beforehand. Then, image-assisted navigation via the screen 17 can be provided to a person carrying out the treatment.

The navigation system 13 also can fulfil other tasks. Via a transmitter 18, the surgical saw can thus transmit information about the load state at the saw blade 3, which can be received by a receiver 19 on the navigation system 13. This load state information then can be processed by the navigation system 13, and information regarding the load state can be output by means of the screen 17. This information can include instructions to reduce the force on the saw (e.g., also on the saw blade 3) or to decrease the oscillation speed. It also can be indicated that, in a subsequent procedure, a bone tissue is to be prepared that requires a lower or higher tool force. Because the load can always be controlled such that the saw blade 3 does not bend, it may in turn be assumed that the functional section of the saw blade, i.e., its sawing front section, is actually situated at the point indicated by the navigation system 13. All these monitoring and control and/or feedback functions allow a precise treatment and therefore serve the good of the patient.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A surgical instrument, comprising:
a holding section;
a tool removably attachable to the holding section; and
at least one load sensor operative to measure a mechanical load exerted by the tool on the instrument.

2. The instrument according to claim 1, wherein the load sensor comprises a force or torque sensor.

3. The instrument according to claim 1, further comprising a load indicator coupled to the surgical instrument, wherein the at least one load sensor is operatively coupled to the load indicator.

4. The instrument according to claim 3, wherein the load indicator is coupled to the holding section.

5. The instrument according to claim 1, wherein the load indicator comprises at least one load state indicator operative to indicate a load state.

6. The instrument according to claim 5, wherein the load indicator is indicates a load state via a color code.

7. The instrument according to claim 1, further comprising a tool adaptor.

8. The instrument according to claim 7, wherein the tool adapter is a universal adaptor operable to accept a number of matching tools.

9. The instrument according to claim 8, wherein the tool adapter is a plug or latch connection.

10. The instrument according to claim 7, wherein the load sensor is situated on the tool adaptor or on a drive member including the tool adaptor.

11. The instrument according to claim 1, wherein the tool comprises an identification device operative to transmit information about a nature of the tool to a receiver on the instrument.

12. The instrument according to claim 11, wherein the receiver is attached to the holding section.

13. The instrument according to claim 11, wherein the identification device is an RFID (radio-frequency identification) transponder and the receiver is an RFID receiver or an RFID transmitter/receiver unit.

14. The instrument according to claim 1, further comprising a tool drive, wherein the load sensor is arranged at a point in a drive chain of the tool.

15. The instrument according to claim 1, further comprising a drive-transmitting coupling arranged on the holding section, wherein the load sensor is situated on the coupling.

16. The instrument according claim 1, wherein the instrument is a surgical, electrically or pneumatically operated saw comprising an oscillating saw blade tool.

17. A medical instrument monitoring system, comprising:
a surgical instrument according to claim 1;
a transmission unit coupled to the surgical instrument; and
a receiver assigned to a data processing unit, wherein the transmission unit is operable to transmit a load state ascertained by the load sensor to the receiver.

18. The system according to claim 17, wherein the data processing unit includes an image output device on which the load state can be output.

19. The system according to claim 17, wherein the data processing unit is assigned to a medical tracking and navigation system operable to determine and track a position of the instrument.

20. The system according to claim 19, wherein the tracking and navigation system includes an evaluation unit that correlates the load state and movement of the instrument to ascertain data about necessary or possible adaptations of the movement or operation of the instrument, and wherein the data is output via the image output.

21. A method of determining a force applied to a tool of a surgical instrument, said surgical instrument including a holding section for operating the surgical instrument, and a tool adapter for coupling the tool to the holding portion, comprising measuring a mechanical load applied by the tool on the instrument.
